(19) United States
(12) Patent Application Publication
(10) Pub. No.: US 2003/0124610 A1
(43) Pub. Date: Jul. 3, 2003

(54) Method for the analysis of a selected multicomponent sample

(75) Inventors: Olav Kvalheim, Bergen (NO); Bjorn Grung, Bergen (NO)

Correspondence Address: BACON & THOMAS, PLLC, 625 SLATERS LANE, FOURTH FLOOR, ALEXANDRIA, VA, 22314

(73) Assignee: Pattern Recognition Systems Holding AS, Bergen (NO)

(21) Appl. No.: 10/335,919

(22) Filed: Jan. 3, 2003

Related U.S. Application Data
(63) Continuation-in-part of application No. PCT/GB01/02960, filed on Jul. 4, 2001.

(30) Foreign Application Priority Data
Jul. 4, 2000 (GB) 0016459.0
Sep. 18, 2002 (GB) 0221702.4

(51) Int. Cl. C12Q 1/68; G01N 33/53; G06F 19/00; G01N 33/48; G01N 33/50

(52) U.S. Cl. 435/6; 435/7.1; 702/19; 702/20

(57) ABSTRACT

The application describes a method for predicting chemical or biological properties, e.g. toxicity, mutagenicity, etc., of complex multicomponent mixtures from 2D separation date, e.g. GC-MS. The data are resolved into peaks (C) and spectra (S) for individual components by an automated curve resolution procedure (GENTLE). The resolved peaks are then integrated and the characteristic area, separation parameter and associated spectrum combined to yield a predictor matrix (X), which is used as input to a multivariate regression model. Partial least squares (PLS) are used to correlate the 2D separation date for a training set to the measured property. The regression model can then be used to predict the property for other samples.

This invention relates to a method of analysis of data, in particular data from systems having a large number of components, for example compositions containing large numbers of unidentified chemical compounds, and to programs and computers arranged to perform such analysis.

In environmental monitoring and medical diagnostic assaying, the analyst may be provided with samples (for example body fluids or liquid or gaseous effluent samples) containing large numbers of unidentified chemical or biological components, for example hundreds of chemical compounds, and required to determine whether the material sampled poses an environmental risk or contains evidence of a disease state. One typical technique used is the so-called Ames test in which a selected mutant strain of a bacterium is exposed to the sample and the toxicity (mutagenicity) of an environmental sample is assessed by determining the extent to which the bacterium is mutated to possess characteristics present in the natural (wild) strain of the bacterium but absent in the selected mutated strain.

It will be appreciated that such a test simply provides an indication of the toxicity of the particular sample and gives no indication of the particular compound or compounds responsible for the toxicity and gives no basis for predicting the toxicity of other samples.

Likewise most diagnostic assays simply detect the presence or abundance of a single compound and give no indication of the presence or abundance of other compounds which may also be indicative of the particular disease state or other disease states.

Chromatographic techniques, e.g. liquid or gas chromatography, may be used to separate individual components of a multicomponent mixture, and spectroscopic techniques, e.g. mass spectroscopy, IR, UV, Raman, ESR and NMR spectroscopy can be used to determine spectra characteristic of such individual components; however chromatographic separation is normally not capable of isolating each individual component of a mixture of hundreds of chemical compounds and it is expensive, time-consuming and generally impractical to carry out separate toxicity or other tests on all fractions or components of a multicomponent sample.

There thus exists a need for a method for analysis of multicomponent mixtures which is capable of being used to predict an effect (e.g. toxicity) of the mixture as a whole and to focus down on and perhaps identify the components having a major contribution to that effect.

More especially there is a need for such a method wherein it is not necessary to identify in advance the components of the mixture which are or are thought to be responsible for the beneficial or detrimental properties of the mixture.

It has now been found that such a method is capable of being put into effect where, for a plurality of similar samples, data is available for the effect of the samples and characteristic spectroscopic data is available for separated fractions of the samples, e.g. chromatographically separated fractions of the samples.

Thus viewed from one aspect the present invention provides a method for the analysis of a selected multicomponent sample to predict a value of a property thereof, which method comprises:

i) determining a value of said property for a plurality of similar multicomponent samples;

ii) for each said similar sample,

a) separating the components thereof along a separation dimension,

b) sampling portions thereof at a plurality of positions along said separation dimension,

c) determining a pattern for each portion which is characteristic of its single or multicomponent nature,

d) selecting sets of said patterns for sections of said separation dimension and determining therefrom patterns and separation dimension profiles characteristic of individual components in said portions;

iii) comparing the determined patterns and their profiles' positions along the separation dimension whereby to identify analogous components in said similar samples;

iv) comparing the values of said property and the intensities of the determined profiles for components in said similar samples whereby to generate a model predictive of the value of said property for a sample; and

v) for said selected sample,

A) separating the components thereof along a separation dimension,

B) sampling portions thereof at a plurality of positions along said separation dimension,

C) determining a pattern for each portion which is characteristic of its single or multicomponent nature,

D) selecting sets of said patterns for sections of said separation dimension and determining therefrom patterns and separation dimension profiles characteristic of individual components in the portions; and

E) applying said model to the intensities of determined profiles for components in said selected sample whereby to generate an estimate of the value of said property for said selected sample.

The “property” referred to may be any one capable of being assigned a numerical value; however this may for example be zero or one where the property is one where no intermediate gradation is possible or necessary, e.g. dead or alive, infected or not infected, etc.

Preferably, the step (d) of selecting sets of said patterns for sections of said separation dimension is carried out other than by way of predetermined chemical identities of components in said samples. Thus, the selection is made without prior knowledge of the chemical identities of all the compounds contributing to the property of the multicomponent sample. The identities of one or some of the compounds which contribute to the property may of course be known and in some instances it may of course turn out that with certain samples the compounds contributing to the property were known. The method described herein however does not require prior knowledge of the compounds contributing to the property and does not require those compounds to be identifiable by database comparison or other searching techniques.

The method of the invention involves building a prediction model based on the analysis of similar samples for which a value of the property has been determined and then applying this model to the analysis results for a sample for which the property need not be determined. By similar is meant that the samples are of the same type and come from the same or similar type of source, e.g. the samples are all gaseous or liquid effluents from the same process or operation or are derived from the same body fluid, tissue, exudate, etc. from members of the same species, for example blood, serum, plasma, urine, mucous, sputum, faeces, swat, body gases, or body tissue or from members of the same plant or microorganism genus or species, etc. Thus the “similar” samples will together contain a plurality of, and preferably all or the majority of, the components present in the “selected” sample.

The method of the invention involves separating individual components of the multicomponent samples. Such separation may be but need not be complete and each portion which is sampled (for example for mass spectral, nmr or other spectral analysis, e.g. UV, IR, raman, esr, etc.) may thus contain one or more components. Thus if the separation is by means of gas or liquid chromatography, the same component may be present in several neighbouring portions along the separation dimension (e.g. elution time). The method as applied to gas chromatography-mass spectroscopy (GC-MS) thus involves investigating the MS spectra for neighbouring portions so as to identify MS peaks characteristic of individual components and calculate the GC profiles along elution time of those individual components. If desired, data for uninteresting sections of the separation dimension may be discarded and so the components for which profiles are determined may only need to comprise a subset of the total number of components present. The intensities (e.g. peak heights or peak areas or simply a yes/no value) of those determined profiles are used for the construction and application of the prediction model. The prediction model is made accurate by comparing the data for the different samples to identify as analogous components which are identical or closely similar in terms of profile (e.g. retention time or adjusted retention time) and pattern (e.g. mass spectrum).

For the analysis of many samples it will be feasible for a supplier to provide the user with a pre-calculated prediction model, thus viewed from a further aspect the invention provides a method for the production of a prediction model for predicting a value of a property of a multicomponent sample, which method comprises:

i) determining a value of said property for a plurality of similar multicomponent samples;

ii) for each said similar sample,

a) separating the components thereof along a separation dimension,

b) sampling portions thereof at a plurality of positions along said separation dimension,

c) determining a pattern for each portion which is characteristic of its single or multicomponent nature,

d) selecting sets of said patterns for sections of said separation dimension and determining therefrom patterns and separation dimension profiles characteristic of individual components in said portions;

iii) comparing the determined patterns and their profiles' positions along the separation dimension whereby to identify analogous components in said similar samples; and

iv) comparing the values of said property and the intensities of the determined profiles for components in said similar samples whereby to generate a model predictive of the value of said property for a sample.

Preferably, the step (d) of selecting sets of said patterns for sections of said separation dimension is carried out other than by way of predetermined chemical identities of components in said samples.

Viewed from a still further aspect the invention provides a method for the analysis of a selected multicomponent sample to predict a value of a property thereof, which method comprises:

A) separating the components thereof along a separation dimension,

B) sampling portions thereof at a plurality of positions along said separation dimension,

C) determining a pattern for each portion which is characteristic of its single or multicomponent nature,

D) selecting sets of said patterns for sections of said separation dimension and determining therefrom patterns and separation dimension profiles characteristic of individual components in the portions, and

E) applying a prediction model to the intensities of determined profiles for components in said selected sample whereby to generate an estimate of the value of said property for said selected sample.

Preferably, the step (D) of selecting sets of said patterns for sections of said separation dimension is carried out other than by way of predetermined chemical identities of components in said sample.

The methods of the invention may be used to predict the properties of a multicomponent sample without requiring the determination of the identities of the components in or likely to be in the sample. The method is thus of particular use in the quality control of multicomponent samples of biological origin, especially of plant, bacterial, fungal or animal origin, particularly plant extracts and other materials used as phytopharmaceuticals, nutraceuticals or traditional medicines. Viewed from a further aspect therefore the invention provides a material (e.g. plant or plant extract, phytopharmaceutical, nutraceutical or traditional medicine), for example a batch of a material, quality controlled by a method according to the invention. The material quality controlled in this manner may be one where the quality control is to determine whether the material is suitable for consumption or use or one where the quality control is to determine whether the material (e.g. effluent) is safe or toxic.

Quality control according to the invention may involve operation of a simple pass/fail criterion whereby a sample is passed or failed if compounds identified by the prediction model are present at concentrations above or below a particular threshold value.

The analysis of biological material according to the invention may particularly preferably be effected using samples taken from different geographic locations, of different species, collected at different growth stages, grown in different soil types, collected at different times of the year, stored or transported under different conditions, etc. In this way, the methods of the invention may be used to identify optimum sources, growth and harvesting conditions, storage conditions, etc, as well as to predict whether or not a particular batch of such a sample meets quality control criteria.

The methods of the invention may also be used in the identification of biologically active agents, e.g. drug substances, and combinations thereof. Thus a complex mixture with a desired effect can be screened using the method of the invention to identify which positions on the separation and spectral analysis axes correspond to components responsible for or contributing to the desired effect. Fractions of the sample from those separation positions may be analysed to identify the relevant components.

In one preferred embodiment, fractions of the sample(s) from the relevant separation positions may then be subjected to a repetition of the method of the invention using a different separation technique (e.g. liquid chromatography) and if desired a different spectral analysis (e.g. nmr or diode array detection rather than MS). In this way identification of the active components is further facilitated. Likewise, where a synergistic or complimentary action involving two or more components of the sample is responsible for the desired property, these components may readily be identified using the methods of the invention. In this event, it is particularly useful to use as the “training” samples for the method, samples produced from source material using different extraction or separation techniques, and mixtures thereof in different ratios. In this way the “training” samples themselves will serve to narrow down the list of possible identities for the active component making their identification and/or isolation much simpler to achieve.

Thus viewed from a further aspect the invention provides a method for the identification of a biologically active component or component combination in a material having a desired or undesired property, which method comprises:

i) determining a value of said property for a plurality of trial samples of said material of different chemical composition;

ii) for each said trial sample,

a) separating the components thereof along a separation dimension,

b) sampling portions thereof at a plurality of positions along said separation dimension,

c) determining a pattern for each portion which is characteristic of its single or multicomponent nature,

d) other than by way of predetermined chemical identities of components in said samples, selecting sets of said patterns for sections of said separation dimension and determining therefrom patterns and separation dimension profiles characteristic of individual components in said portions;

iii) comparing the determined patterns and their profiles' positions along the separation dimension whereby to identify analogous components in said trial samples;

iv) comparing the values of said property and the intensities of the determined profiles for components in said trial samples whereby to generate a model predictive of the active component or component combination in said source material;

v) chemically identifying said active component or component combination, and optionally synthesizing said active component or component combination or a derivative thereof and optionally formulating the synthesised said active component or component combination, and optionally selecting a source of said material and optionally formulating said material from said source or an extract therefrom containing said active component or component combination.

In this context, biological activity may be a desirable property (e.g. the compounds may be useful in therapy or prophylaxis) or an undesirable property (e.g. the compounds may be toxic). Thus the method can be used to identify pharmacologically useful compounds in plants or toxic chemicals or biological markers for toxic chemicals in effluent or environmental samples, in foodstuffs, etc.

Chemical identification in this method will generally be effected in conventional fashion using a combination of available analytical techniques, e.g. chromatographic separation, nmr, ms, ir, uv, raman, esr spectroscopy, atomic analysis, X-ray crystallography, etc. Derivatization may for example involve salt formation, amino acid substitution, addition or deletion, addition of functional groups to increase hydrophilicity or lipophilicity, attachment of biodistribution modifying moieties, etc.

While, as will be discussed further below, the methods of the invention are more broadly applicable to multicomponent samples, the methods will be described in further detail in relation to the analysis of samples containing a plurality of chemical compounds for quantifiable properties such as physical, chemical and more especially biological properties (e.g. toxicity, mutagenicity, disease state, genotype, therapeutic effect, etc) using chromatographic separation to produce the portions and spectroscopic analysis to produce the patterns.

Although, as mentioned above, many varieties of spectroscopic analysis may be used, techniques in which the spectroscopic peaks (or troughs) are sharp are specially preferred, e.g. nmr or more especially mass spectroscopy (ms). Likewise separation is preferably performed using liquid or more preferably gas chromatography.

Equipment is available which can generate chromatographically separated spectroscopic data for samples, e.g. GC-MS apparatus.

Thus the starting data for the analysis according to the invention may be considered to be a two-dimensional matrix (i.e. chromatographic portion data, and spectroscopic data for each chromatographic portion) together with determined property values for each sample for the generation of the prediction model and a two-dimensional matrix for the generation of a predicted value for a selected sample (i.e. chromatographic portion data, and spectroscopic data for each chromatographic portion). Likewise, the chromatographic and spectrographic data will contain intensity and position (e.g. elution time or mass number or m/e ratio) data.

To reduce the required computing time, which is particularly important where the number of compounds in the samples is in the hundreds, the input data may be restricted by removing data where the height is below a pre-set minimum (e.g. where the amount of compounds from the sample in the fraction is nil or very low or where the spectroscopic peak is at noise level) or where the portion corresponds to compounds known or thought to have no effect on the property (e.g. low molecular weight, rapidly eluting compounds).

Generally the data matrix is first reduced by discarding data for elution times at which no components elute, i.e. where the chromatographic signal (height) is below a pre-set limit. However, the cut is preferably made at a position along the time direction at which the signal is small relative to the peak height.

This may be achieved by setting a neighbour peak ratio value, e.g. of 0.1 to 0.4, preferably 0.3, and only cutting when the ratio of signal to peak is below this value rather than at the time position at which the signal reaches a minimum following the peak or at the time position at which the signal gets below the pre-set cut limit. The cut limit itself will generally be set according to the needs of the user—a higher value discards more data thus ignoring more minor components and vice versa. Typically it might be set at 5 to 10% of the minimum distinct signal height. Obviously, the lower the cut limit the more data will be retained and the more components will be analysed for.

2D GC-MS data can contain background noise for a variety of reasons. Changes in detector performance can lead to offset and drift in the chromatographic baseline, and column bleeding can lead to the presence of a background spectrum. This makes it desirable to perform a background correction on the chromatographic peaks remaining after discarding the zero signal or noise signal retention times. This may be done by calculating a first order (i.e. linear) estimated baseline having a slope approximating the slope of a line extrapolated from the zero component regions on either side of the peak cluster.

For each chromatogram peak cluster selected in this way, the separate spectroscopic data sets can be normalized, e.g. setting maximum spectral peak height to 1 or overall spectroscopic peaks area to 1, or to a value proportional to that peak area of the selected chromatographic peak cluster.

Preferably, chromatographic peak clusters selected in this way extend over at least 20 resolution time valves, i.e. they have associated with them at least 20 ms spectra.

Data reduction of the spectral data can then likewise be performed. Thus, for MS, if one considers the whole elution time at once, most or even all of the mass numbers in the recordable range contain a signal from at least one component. In the mass spectra for chromatogram portions however, many mass numbers contain no signal or signal due only to noise. The presence of such mass numbers reduces the quality of the resolution process and they are preferably removed from the spectra prior to resolution.

While it is trivial to detect mass numbers with zero signal, mass numbers with a signal due to random noise can be detected by using a morphological criterion in combination with an F-test (see Shen et al. Chemomem. Intell. Lab. Syst. 51: 37-47 (2000)) which utilizes the fact that noise has a higher frequency than signal from a chemical component. In this way, up to about 90% of the mass spectral data may be discarded prior to resolution.

The adjusted spectral data can then be resolved into individual peaks. This effectively involves solving the equation

X=CS^{T}+E (1)

for C and S, wherein X is the recorded data, C is the chromatographic profiles, S is the mass spectra, T denotes a matrix transpose and E is the residual matrix.

This may be done in many ways. However, one preferred way is the GENTLE method described by Manne et al in Chemom. Intell. Lab. Syst. 50: 35-46 (2000), the contents of which are hereby incorporated by reference.

First A key spectra Sₒ are found, e.g. using a simplified Borgen method (see Grande et al., Chemom. Intell. Lab. Syst. 50: 19-33 (2000), the contents of which are incorporated by reference). (“A” here is the chemical rank). In a peak cluster the key spectra are the purest spectra. The key spectra are found by normalizing the data to constant projection on the first singular vector of the data. (The term “singular” implies that the vector is the result of a singular value decomposition (SVD), which is a standard numerical method. In matrix form, X=UΣV^{T}. The first column vector of U, sometimes referred to as the first left singular vector, is used for the projection.) The key spectra can then be found on extreme points on the convex and bounded representation of the data that thus appears. The key spectra Sₒ represent initial estimates of the true spectra S. Initial estimation Cₒ of the true chromatographic profiles Cₒ can then be found by solving equation (1) for C, thus

Cₒ=XSₒ (Sₒ^{T}Sₒ)⁻¹ (2)

To obtain estimates of true profiles and spectra, C and S, from the initial estimates Cₒ and Sₒ, an iterative procedure is invoked. This may be done by determining a transformation matrix T for which equations (3) and (4) hold:

C=CₒT (3)

S^{T}=T⁻¹Sₒ^{T} (4)

T is the product of several elementary matrices and may be generated by an iterative approach which is facilitated by placing certain constraints on the intermediate solutions for C and S. Thus for S and C it is presumed that a peak (whether in the chromatograph or the mass spectra) must be positive and for C it is presumed that a pure chromatographic peak should be unimodal. The following criteria may for example be used to achieve and evaluate the resolution:

Component windows: linear regression may be used to minimize the non-zero deviation for a component outside the chromatographic region where it is above the noise limit.

Smoothness: the chromatographic peak for a compound may be assumed to be continuous (thus distinguishing it from noise).

Significance: the apex intensity of the chromatographic peak for a component should generally be significantly higher than the decision limit for the data (i.e. the cut limit or minimum distinct signal height referred to earlier); typically peaks should only be accepted if their apex intensity is at least twice the decision limit.

Integrity: a check is preferably made that a resolved peak decreases to noise level before the selected chromatographic peak cluster ends; if it does not, the procedure should be repeated with a larger peak cluster.

The chemical rank, or the number of key spectra to be found may be found iteratively, starting with a relatively large number, e.g. 8 to 12, preferably 10. After calculating a solution according to the particular number of key spectra, the solutions are evaluated according to the criteria above. If the quality of the resolved profiles is poor, resolution is repeated with a larger or, more generally, smaller number of key spectra.

After resolution, the resolved mass spectra S may be normalised so that maximum intensity is 1.0 and the chromatographic profiles C can be recalculated as:

C=XS(S^{T}S)⁻¹ (5)

The qualitative information is then present in the spectra while the quantitative information is present in the chromatographic profiles (which are integratable to provide an area).

In effect the resolution procedure involves a comparison of the selected mass spectra for a sample to identify groups of spectral lines characteristic of the individual chemical components in the sample and determination of characteristic chromatographic profiles for such components. The output data for a sample is then a list of individual components, characterised by the mass spectral lines and by the position (i.e. elution time) and the area of their chromatographic profiles. With this done for a plurality of samples, a predictor matrix can be generated and this may be used to generate a predictor model. Thus for example Y=Xb, where X is predictor matrix, b are the regression coefficients (the predictor model) and Y is the predicted values of the sample property.

Thus, in the generation of the predictor matrix, the output data for the different samples is compared and the presence of similar components (i.e. chemical compounds) is determined. Regression analysis can then be used to determine the relative magnitude and negative or positive nature of the contribution of each component to the overall measured property (e.g. carcinogenicity) of the samples. These contributions can then be expressed as a predictor model of the contribution for each component. By applying this predictor model to the determined component concentration profile for a further sample, a value for the property for the further sample can then be estimated simply.

Typically, the production of the predictor matrix involves the following steps:

i) loading of the resolved profiles for the samples for which a value of the property has been measured, the profile for each example typically comprising an area (the chromatographic peak area), a retention time and a normalized mass spectrum for each resolved component;

ii) sorting the resolved profiles in order of increasing retention time;

iii) comparing the mass spectra for different components which have a retention time within a selected range, e.g. 1 to 8 minutes, typically 4 minutes, so as to identify components which are common to two or more samples thereby reducing the number of variables for the subsequent regression analysis; and

iv) establishing a regression model correlating measured values of the property to the sets of values of retention time and area for the resolved components of the samples.

The comparison step (iii) typically involves determination of a spectral similarity index Sᵢⱼ between the mass spectra Sᵢ and Sⱼ of components i and j in different samples but with similar retention times. Sᵢⱼ can be expressed as:

Sᵢⱼ=Sᵢ^{T}·Sⱼ (6)

and if it has a value above a pre set limit (e.g. 0.9) the components i and j can be classified as analogous.

When the predictor matrix has been established, a classification model or regression model is estimated correlating measured values of the property to the sets of areas calculated for the resolved components of the samples. The calculation of the model from the predictor matrix can be effected by commercially available multivariant classification/regression analysis computer programs, e.g. the program Sirius available from Pattern Recognition Systems AS of Bergen, Norway.

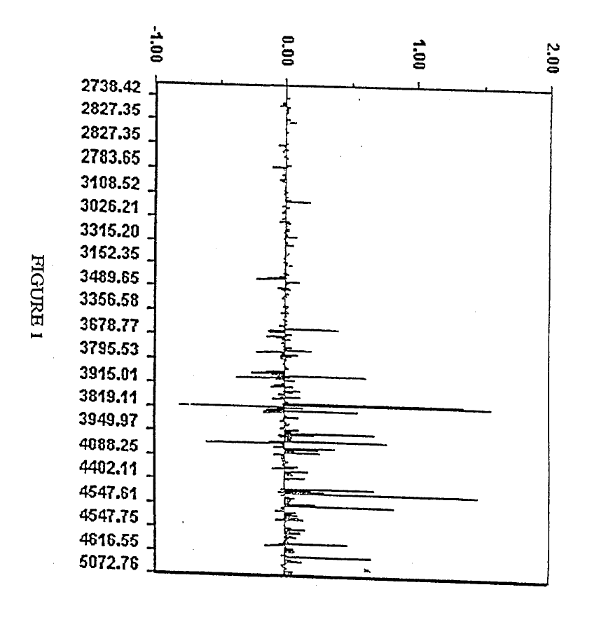

An example of a typical prediction model is shown schematically in FIG. 1 of the accompanying drawings. In this figure, the x axis is component retention time while the y axis is the value of the regression coefficient for each of the components resolved in the samples for which the property was measured. In this case, the property measured was mutagenicity (measured using the Ames test), and the samples were environmental effluent samples.

The biological impact is greater for the components with larger values of regression coefficient and, as can be seen, these tended to be components with larger retention times.

The comparison step may if desired be facilitated by spiking the samples before GC-MS analysis with chemical compounds with known mass spectra which would not otherwise have been present in the samples. Any variation in the retention times for these compounds can be used to decide the size of the selected range of retention times over which analogous compounds are determined. The profiles for those spiking compounds would not however be used in the generation of the predictor matrix since, not being present in the unspiked samples, they clearly cannot contribute to the value of the property. Moreover the spiking can be used to allow compensation for variations between samples in the quantity of sample injected into the GC-MS, i.e. the peak areas may be normalized relative to the peak area of the spiking agent.

While the discussion above has mainly been in terms of correlation of GC-MS spectra of multicomponent chemical samples with a measurable value of biological impact, the methods of the invention are more generally applicable. Thus for example they may be used to test food samples for biological or chemical contamination, e.g. by toxins such as DSP, PSP, ASP, aflatoxins and botulinum toxin, or for analysis of medical samples, e.g. lymph, blood, serum, plasma, urine, mucous, semen, sputum, faeces or tissue samples, to detect conditions such as bacterial and viral infections, prion-related diseases, physiological conditions such as Alzheimer's disease, whiplash, etc. or substance abuse (e.g. use of illegal drugs or use of proscribed substances by athletes). The methods however are generally applicable to any system where a measurable property can be correlated to a “signature” set of signals from a plurality of components.

The methods of the invention are particularly applicable to medical and forensic diagnosis. Thus in one embodiment the “property” may be normal/healthy or abnormal/unhealthy, using as the sample a body tissue or fluid (e.g. blood, plasma or serum), and components may be identified as correlating with abnormality or ill health or as correlating with abnormality or ill health if they are present outside a particular concentration range. Similarly components or sets of components may be identified as correlating with particular abnormalities or disease states. In another embodiment, body fluids, tissues or gases may be analysed for time after death and the resultant predictor model used to determine time of death, for example for murder victims.

Equally the methods are especially applicable for testing of foodstuffs (e.g. cheese) to detect abnormality or contamination (either chemical or biological).

If desired, the methods of the invention may be extended to identify one or more of the resolved components of the sample by comparison of the characterising data (e.g. chromatographic profile and/or mass spectrum) of the component with similar characterizing data of known chemicals (or other components), e.g. by cross reference to a computerized data base for a library of chemicals. Thus, the methods of the invention may for example be used as a coarse filter to identify more specific or more precise diagnostic tests which may be applied to a sample (or to further samples from an individual or a test site). In this way a problem may be identified without having to carry out the whole array of available diagnostic tests.

Viewed from a further aspect the invention provides a computer software product (e.g. a disc, tape, wire or memory device or other carrier) carrying a computer program for performing a method according to the invention.

Viewed from a still further aspect the invention provides a computer programmed to perform a method according to the invention.

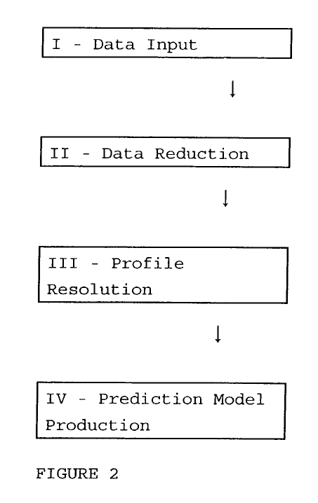

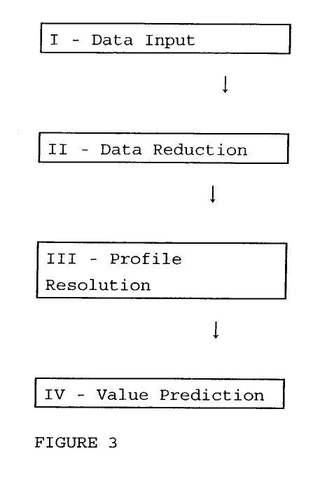

The operation of a program according to the invention is illustrated schematically in the flow diagrams of FIGS. 2 and 3 of the accompanying drawings.

Referring to FIG. 2, the creation of a prediction model is illustrated. Data input (step I) involves loading of GC-MS data and measured property values for a plurality of samples. Data reduction (step II) involves discarding of blank retention times and removal of the background (i.e. identification of GC peak clusters), discarding of blank mass numbers and removal of MS background (i.e. identification of sets of mass spectral peaks from the mass spectra for each GC peak cluster). Profile resolution (step III) involves identifying the mass spectra for individual components in such a GC peak cluster and determining a GC profile (peak retention time and peak area) for each resolved component. Prediction model production (step IV) involves comparison of resolved component profiles between the different samples to identify components common to two or more samples and regression analysis to provide for each resolved component a regression coefficient indicative of the impact of that component on the measured property and production of the prediction model from the resultant predictor matrix.

Referring to FIG. 3, the application of a predictor model is illustrated. Data input (step I) involves loading of GC-MS data for a sample. Data reduction (step II) and profile resolution (step III) are as described for FIG. 2. Value prediction (step IV) involves application of a precalculated prediction model to that resolved profile. It will be clear therefore that only those components used in the construction of the prediction model will be taken account of in the determination of the estimated value of the property.

As mentioned earlier, the prediction model need not be derived based on regression coefficients indicative of component contribution to property but may reflect a classification, i.e. alive/dead, healthy/unhealthy, so that application of the model gives a corresponding classification of the source of the sample as the estimated property value.

It will also be appreciated that the predictor matrix may be used for the data reduction in the production of a predicted value for a sample; thus for example GC retention times corresponding to low values of regression coefficients determined in calculating the predictor matrix may be discarded.

It will be appreciated that the analysis of the invention could be carried out by data processing means located remotely. Thus, from a further aspect the invention provides a computer program product containing instructions which when carried out on data processing means will predict a value of a property of a selected multicomponent sample, wherein the computer program receives data obtained by:

A) separating the components of the sample along a separation dimension; and

B) sampling portions thereof at a plurality of positions along said separation dimension, and wherein the computer program carries out the steps of:

a) determining a pattern for each portion which is characteristic of its single or multicomponent nature;

b) selecting sets of said patterns for sections of said separation dimension and determining therefrom patterns and separation dimension profiles characteristic of individual components in the portions; and

c) applying a prediction model to the intensities of determined profiles for components in said selected sample whereby to generate an estimate of the value of said property for said selected sample.

Preferably step (b) is carried out other than by way of predetermined chemical identities of components in said sample.

From a further aspect the present invention provides a computer program product containing instructions which when carried out on data processing means will analyse a selected multicomponent sample to predict a value of a property thereof, wherein the computer program receives data obtained by:

i) determining a value of said property for a plurality of similar multicomponent samples;

ii) for each said similar sample,

a) separating the components thereof along a separation dimension,

b) sampling portions thereof at a plurality of positions along said separation dimension, and

iii) for said selected sample,

A) separating the components thereof along a separation dimension,

B) sampling portions thereof at a plurality of positions along said separation dimension,

wherein the computer program carries out the steps of:

i) for each said similar sample,

a) determining a pattern for each portion which is characteristic of its single or multicomponent nature, and

b) selecting sets of said patterns for sections of said separation dimension and determining therefrom patterns and separation dimension profiles characteristic of individual components in said portions;

ii) comparing the determined patterns and their profiles' positions along the separation dimension whereby to identify analogous components in said similar samples;

iii) comparing the values of said property and the intensities of the determined profiles for components in said similar samples whereby to generate a model predictive of the value of said property for a sample; and

iv) for said selected sample,

A) determining a pattern for each portion which is characteristic of its single or multicomponent nature,

B) selecting sets of said patterns for sections of said separation dimension and determining therefrom patterns and separation dimension profiles characteristic of individual components in the portions; and

C) applying said model to the intensities of determined profiles for components in said selected sample whereby to generate an estimate of the value of said property for said selected sample.

Preferably the step (b) is carried out other than by way of predetermined chemical identities of components in said samples.

From a still further aspect the present invention provides a computer program product containing instructions which when carried out on data processing means will produce a prediction model for predicting the value of a property of a multicomponent sample, wherein the computer program receives data obtained by:

i) determining a value of said property for a plurality of similar multicomponent samples;

ii) for each said similar sample,

a) separating the components thereof along a separation dimension, and

b) sampling portions thereof at a plurality of positions along said separation dimension, and

wherein the computer program carries out the steps of:

i) for each said similar sample

A) determining a pattern for each portion which is characteristic of its single or multicomponent nature,

B) selecting sets of said patterns for sections of said separation dimension and determining therefrom patterns and separation dimension profiles characteristic of individual components in said portions;

ii) comparing the determined patterns and their profiles' positions along the separation dimension whereby to identify analogous components in said similar samples; and

iii) comparing the values of said property and the intensities of the determined profiles for components in said similar samples whereby to generate a model predictive of the value of said property for a sample.

The step (B) is preferably carried out other than by way of predetermined chemical identities of components in said samples.

The invention further extends to a computer program product containing instructions which when carried out on data processing means will create a computer program product as described above.

1. A method for the analysis of a selected multicomponent sample to predict a value of a property thereof, which method comprises:
i) determining a value of said property for a plurality of similar multicomponent samples;
ii) for each said similar sample,
a) separating the components thereof along a separation dimension,
b) sampling portions thereof at a plurality of positions along said separation dimension,
c) determining a pattern for each portion which is characteristic of its single or multicomponent nature,
d) selecting sets of said patterns for sections of said separation dimension and determining therefrom patterns and separation dimension profiles characteristic of individual components in said portions;
iii) comparing the determined patterns and their profiles' positions along the separation dimension whereby to identify analogous components in said similar samples;
iv) comparing the values of said property and the intensities of the determined profiles for components in said similar samples whereby to generate a model predictive of the value of said property for a sample; and
v) for said selected sample,
A) separating the components thereof along a separation dimension,
B) sampling portions thereof at a plurality of positions along said separation dimension,
C) determining a pattern for each portion which is characteristic of its single or multicomponent nature,
D) selecting sets of said patterns for sections of said separation dimension and determining therefrom patterns and separation dimension profiles characteristic of individual components in the portions; and
E) applying said model to the intensities of determined profiles for components in said selected sample whereby to generate an estimate of the value of said property for said selected sample.

2. A method for the production of a prediction model for predicting the value of a property of a multicomponent sample, which method comprises:
i) determining a value of said property for a plurality of similar multicomponent samples;
ii) for each said similar sample,
a) separating the components thereof along a separation dimension,
b) sampling portions thereof at a plurality of positions along said separation dimension,
c) determining a pattern for each portion which is characteristic of its single or multicomponent nature,
d) selecting sets of said patterns for sections of said separation dimension and determining therefrom patterns and separation dimension profiles characteristic of individual components in said portions;
iii) comparing the determined patterns and their profiles' positions along the separation dimension whereby to identify analogous components in said similar samples; and
iv) comparing the values of said property and the intensities of the determined profiles for components in said similar samples whereby to generate a model predictive of the value of said property for a sample.

3. A method for the analysis of a selected multicomponent sample to predict a value of a property thereof, which method comprises:
A) separating the components thereof along a separation dimension,
B) sampling portions thereof at a plurality of positions along said separation dimension,
C) determining a pattern for each portion which is characteristic of its single or multicomponent nature,
D) selecting sets of said patterns for sections of said separation dimension and determining therefrom patterns and separation dimension profiles characteristic of individual components in the portions, and
E) applying a prediction model to the intensities of determined profiles for components in said selected sample whereby to generate an estimate of the value of said property for said selected sample.

4. A method as claimed in claim 1 wherein said samples are compositions containing a plurality of different chemical or biological components, and separation of said samples is effected chromatographically.

5. A method as claimed in claim 2 wherein said samples are compositions containing a plurality of different chemical or biological components, and separation of said samples is effected chromatographically.

6. A method as claimed in claim 3 wherein said samples are compositions containing a plurality of different chemical or biological components, and separation of said samples is effected chromatographically.

7. A method as claimed in claim 4 wherein said patterns are spectrographic patterns.

8. A method as claimed in claim 5 wherein said patterns are spectrographic patterns.

9. A method as claimed in claim 6 wherein said patterns are spectrographic patterns.

10. A method as claimed in claim 4 wherein said samples are or derive from body tissue or fluids or exudates or are or derive from environmental fluids, and separation is effected by gas or liquid chromatography.

11. A method as claimed in claim 5 wherein said samples are or derive from body tissue or fluids or exudates or are or derive from environmental fluids, and separation is effected by gas or liquid chromatography.

12. A method as claimed in claim 6 wherein said samples are or derive from body tissue or fluids or exudates or are or derive from environmental fluids, and separation is effected by gas or liquid chromatography.

13. A method as claimed in claim 4 wherein said patterns are mass spectra.

14. A method as claimed in claim 5 wherein said patterns are mass spectra.

15. A method as claimed in claim 6 wherein said patterns are mass spectra.

16. A method as claimed in claim 1 wherein in step d, sets of said patterns are selected other than by way of predetermined chemical identities of components in said samples.

17. A method as claimed in claim 2 wherein in step d, sets of said patterns are selected other than by way of predetermined chemical identities of components in said samples.

18. A method as claimed in claim 3 wherein in step D sets of said patterns are selected other than by way of predetermined chemical identities of components in said samples.

19. A method as claimed in claim 1, wherein said sets of patterns are selected so as to discard sections of said separation dimension for which the sampling signal obtained is below a predetermined level.

20. A method as claimed in claim 2, wherein said sets of patterns are selected so as to discard sections of said separation dimension for which the sampling signal obtained is below a predetermined level.

21. A method as claimed in claim 3, wherein said sets of patterns are selected so as to discard sections of said separation dimension for which the sampling signal obtained is below a predetermined level.

22. A method as claimed in claim 19, wherein only sections of said separation dimension for which the ratio of the signal level of the sampled portion to the signal level of the nearest peak along the separation dimension is less than between 0.1 and 0.4 are discarded.

23. A method as claimed in claim 20, wherein only sections of said separation dimension for which the ratio of the signal level of the sampled portion to the signal level of the nearest peak along the separation dimension is less than between 0.1 and 0.4 are discarded.

24. A method as claimed in claim 21, wherein only sections of said separation dimension for which the ratio of the signal level of the sampled portion to the signal level of the nearest peak along the separation dimension is less than between 0.1 and 0.4 are discarded.

25. A method as claimed in claim 22, wherein only sections of said separation dimension for which the ratio of the signal level of the sampled portion to the signal level of the nearest peak along the separation dimension is less than 0.3 are discarded.

26. A method as claimed in claim 1, wherein said sets of patterns are selected so as to discard sections of said separation dimension relating to components which are known or thought to have little or no effect on said property.

27. A method as claimed in claim 2, wherein said sets of patterns are selected so as to discard sections of said separation dimension relating to components which are known or thought to have little or no effect on said property.

28. A method as claimed in claim 3, wherein said sets of patterns are selected so as to discard sections of said separation dimension relating to components which are known or thought to have little or no effect on said property.

29. A method as claimed in claim 1, wherein said selected sets of patterns for said separation dimension are corrected for background noise.

30. A method as claimed in claim 2, wherein said selected sets of patterns for said separation dimension are corrected for background noise.

31. A method as claimed in claim 3, wherein said selected sets of patterns for said separation dimension are corrected for background noise.

32. A method as claimed in claim 7, wherein the spectral data in the selected patterns which contains no signal or only a signal due to noise is discarded.

33. A method as claimed in claim 8, wherein the spectral data in the selected patterns which contains no signal or only a signal due to noise is discarded.

34. A method as claimed in claim 9, wherein the spectral data in the selected patterns which contains no signal or only a signal due to noise is discarded.

35. A method as claimed in claim 7, wherein the spectral patterns obtained are resolved into individual peaks using the Gentle method.

36. A computer software product for performing a method according to claim 1.

37. A computer software product for performing a method according to claim 2.

38. A computer software product for performing a method according to claim 3.

39. A computer programmed to perform a method according to claim 1.

40. A computer programmed to perform a method according to claim 2.

41. A computer programmed to perform a method according to claim 3.

42. A computer program product containing instructions which when carried out on data processing means will predict a value of a property of a selected multicomponent sample, wherein the computer program receives data obtained by:
A) separating the components of the sample along a separation dimension; and
B) sampling portions thereof at a plurality of positions along said separation dimension, and wherein the computer program carries out the steps of:
a) determining a pattern for each portion which is characteristic of its single or multicomponent nature;
b) selecting sets of said patterns for sections of said separation dimension and determining therefrom patterns and separation dimension profiles characteristic of individual components in the portions; and
c) applying a prediction model to the intensities of determined profiles for components in said selected sample whereby to generate an estimate of the value of said property for said selected sample.

43. A computer program product containing instructions which when carried out on data processing means will analyse a selected multicomponent sample to predict a value of a property thereof, wherein the computer program receives data obtained by:
i) determining a value of said property for a plurality of similar multicomponent samples;
ii) for each said similar sample,
a) separating the components thereof along a separation dimension,
b) sampling portions thereof at a plurality of positions along said separation dimension, and
iii) for said selected sample,
A) separating the components thereof along a separation dimension,
B) sampling portions thereof at a plurality of positions along said separation dimension,
wherein the computer program carries out the steps of:
i) for each said similar sample,
a) determining a pattern for each portion which is characteristic of its single or multicomponent nature,
b) selecting sets of said patterns for sections of said separation dimension and determining therefrom patterns and separation dimension profiles characteristic of individual components in said portions;
ii) comparing the determined patterns and their profiles' positions along the separation dimension whereby to identify analogous components in said similar samples;
iii) comparing the values of said property and the intensities of the determined profiles for components in said similar samples whereby to generate a model predictive of the value of said property for a sample; and
iv) for said selected sample,
determining a pattern for each portion which is characteristic of its single or multicomponent nature,
B) selecting sets of said patterns for sections of said separation dimension and determining therefrom patterns and separation dimension profiles characteristic of individual components in the portions; and
C) applying said model to the intensities of determined profiles for components in said selected sample whereby to generate an estimate of the value of said property for said selected sample.

44. A computer program product containing instructions which when carried out on data processing means will produce a prediction model for predicting the value of a property of a multicomponent sample, wherein the computer program receives data obtained by:
i) determining a value of said property for a plurality of similar multicomponent samples;
ii) for each said similar sample,
a) separating the components thereof along a separation dimension,
b) sampling portions thereof at a plurality of positions along said separation dimension, and
wherein the computer program carries out the steps of:
i) for each said similar sample,
A) determining a pattern for each portion which is characteristic of its single or multicomponent nature,
B) selecting sets of said patterns for sections of said separation dimension and determining therefrom patterns and separation dimension profiles characteristic of individual components in said portions;
ii) comparing the determined patterns and their profiles' positions along the separation dimension whereby to identify analogous components in said similar samples; and
iii) comparing the values of said property and the intensities of the determined profiles for components in said similar samples whereby to generate a model predictive of the value of said property for a sample.

45. A computer program product containing instructions which when carried out on data processing means will create a computer program product or computer software product as claimed in claim 36.

46. A computer program product containing instructions which when carried out on data processing means will create a computer program product or computer software product as claimed in claim 37.

47. A computer program product containing instructions which when carried out on data processing means will create a computer program product or computer software product as claimed in claim 38.

48. A computer program product as claimed in claim 42 wherein step (b) of selecting sets of said patterns is carried out other than by way of predetermined chemical identities of components in the sample.

49. A computer program product as claimed in claim 43 wherein step (b) of selecting sets of said patterns is carried out other than by way of predetermined chemical identities of components in the sample.

50. A computer program product as claimed in claim 44 wherein step (B) of selecting sets of said patterns is carried out other than by way of predetermined chemical identities of components in the sample.

51. The use of a method as claimed in claim 1 for quality control of a material.

52. A material quality controlled by a method as claimed in claim 51.

53. A method for the identification of a biologically active component or component combination in a material having a desired or undesired property, which method comprises:
i) determining a value of said property for a plurality of trial samples of said material of different chemical composition;
ii) for each said trial sample,
a) separating the components thereof along a separation dimension,
b) sampling portions thereof at a plurality of positions along said separation dimension,
c) determining a pattern for each portion which is characteristic of its single or multicomponent nature,
d) other than by way of predetermined chemical identities of components in said samples, selecting sets of said patterns for sections of said separation dimension and determining therefrom patterns and separation dimension profiles characteristic of individual components in said portions;
iii) comparing the determined patterns and their profiles' positions along the separation dimension whereby to identify analogous components in said trial samples;
iv) comparing the values of said property and the intensities of the determined profiles for components in said trial samples whereby to generate a model predictive of the active component or component combination in said source material;
v) chemically identifying said active component or component combination, and optionally synthesizing said active component or component combination or a derivative thereof and optionally formulating the synthesised said active component or component combination, and optionally selecting a source of said material and optionally formulating said material from said source or an extract therefrom containing said active component or component combination.
